(19) United States
(12) Patent Application Publication
(10) Pub. No.: US 2026/0219737 A1
(43) Pub. Date: Jul. 30, 2026

(54) CONTEXT-AWARE GESTURES WITH AN ORAL SENSOR INTERFACE

(71) Applicant: Augmental Technologies Inc., San Francisco, CA (US)

(72) Inventors: Tomas Alfonso Vega Galvez, San Francisco, CA (US); Corten Singer, San Francisco, CA (US); Corbin Halliwill, San Francisco, CA (US); Suraj Raju Gowda, Walnut Creek, CA (US); Oscar Rosello, Encinitas, CA (US)

(73) Assignee: Augmental Technologies Inc., San Francisco, CA (US)

(21) Appl. No.: 19/459,021

(22) Filed: Jan. 26, 2026

Related U.S. Application Data
(63) Continuation-in-part of application No. 18/888,237, filed on Sep. 18, 2024.
Application No. 18/888,237 is a continuation-in-part of application No. 18/099,288, filed on Jan. 20, 2023.
Application No. 18/099,288 is a continuation-in-part of application No. 17/366,186, filed on Jul. 2, 2021, now Pat. No. 11,941,161.
(60) Provisional application No. 63/940,179, filed on Dec. 13, 2025.
(60) Provisional application No. 63/817,481, filed on Jun. 4, 2025.
(60) Provisional application No. 63/749,766, filed on Jan. 27, 2025.
(60) Provisional application No. 63/539,130, filed on Sep. 19, 2023.
(60) Provisional application No. 63/301,501, filed on Jan. 21, 2022.
(60) Provisional application No. 63/162,444, filed on Mar. 17, 2021.
(60) Provisional application No. 63/063,455, filed on Aug. 10, 2020.
(60) Provisional application No. 63/047,946, filed on Jul. 3, 2020.

(51) Int. Cl. G06F 3/01; G01L 19/00; G06F 3/0346; G06F 3/0354

(52) U.S. Cl. CPC G06F 3/017; G01L 19/0092; G06F 3/0346; G06F 3/03543; G01L 2019/0053

(57) ABSTRACT

An oral interface is accessed. The oral sensor interface includes a tongue position sensor (TPS). The TPS detects a tongue movement of the user. The oral sensor interface includes an inertial measurement unit (IMU). The IMU detects one or more head movements of the user. The oral sensor interface includes a barometric pressure transducer. The barometric pressure transducer recognizes the activation gesture. The oral sensor interface enables wireless connectivity to an operating system running on one or more processors. The oral sensor interface enables, with the operating system, oral mouse function control. The oral sensor interface is inserted into a user’s oral cavity. An activation gesture by a user is sensed by the oral sensor interface. The activation gesture selects, within the operating system, a mouse control mode within one or more mouse control modes.

### RELATED APPLICATIONS

This application claims the benefit of U.S. provisional patent applications “Context-Aware Gestures With An Oral Sensor Interface” Ser. No. 63/749,766, filed January 27, 2025, “Conversion Of Low Amplitude Voice Detection To Text” Ser. No. 63/817,481, filed June 4, 2025, “Listening Device For Whisper Speech Detection With Passive Noise Blocking” Ser. No. 63/940,179, filed December 13, 2025, and “Microphone Array For Multimodal Sound Detection With A DSP Pipeline” Ser. No. 63/963,061, filed January 19, 2026.

This application is also a continuation-in-part of U.S. patent application “Silent Intraoral Speech Sensing With Audio Feedback” Ser. No. 18/888,237, filed September 18, 2024, which claims the benefit of U.S. provisional patent application “Silent Intraoral Speech Sensing With Audio Feedback” Ser. No. 63/539,130, filed September 19, 2023.

The U.S. patent application “Silent Intraoral Speech Sensing With Audio Feedback” Ser. No. 18/888,237, filed September 18, 2024, is also a continuation-in-part of U.S. patent application “Intraoral Electronic Sensing for Health Monitoring” Ser. No. 18/099,288, filed January 20, 2023, which claims the benefit of U.S. provisional patent application “Intraoral Electronic Sensing for Health Monitoring” Ser. No. 63/301,501, filed January 21, 2022.

The U.S. patent application “Intraoral Electronic Sensing for Health Monitoring” Ser. No. 18/099,288, filed January 20, 2023 is also a continuation-in-part of U.S. patent application “Data Manipulation Using Remote Augmented Sensing” Ser. No. 17/366,186, filed July 2, 2021, which issued as U.S. Pat. No. 11,941,161 on March 26, 2024, which claims the benefit of U.S. provisional patent applications “Data Manipulation Using Remote Augmented Sensing” Ser. No. 63/047,946, filed July 3, 2020, “Gestural Sensing Using In-Ear Inertial Measurements” Ser. No. 63/063,455, filed August 10, 2020, and “Intraoral Connected Processing Devices” Ser. No. 63/162,444, filed March 17, 2021.

Each of the foregoing applications is hereby incorporated by reference in its entirety.

### FIELD OF ART

This application relates generally to data manipulation and more particularly to context-aware gestures with an oral sensor interface.

### BACKGROUND

A remarkable variety of animals use tools. While humans design, manufacture, and implement tools, non-human animals have been discovered to also find, fashion, and use tools. The non-human tool usage has a notable similarity to the human uses. For example, non-humans can use tools for acquiring water and food. A chimpanzee fashions a stick or twig to extract termites from a termite mound. An otter breaks open clams by cracking the shells on a rock, perched on its stomach. While these uses assist the animals to obtain nourishment, many other uses of tools have been observed. Other animal tool uses include tools for combat and defense, construction, communication, grooming, and recreation, among others. While tool use by mammals has been described, other animal tool users include varieties of birds, insects, fish, and cephalopods. Among birds, crows and ravens have been seen to use twigs to capture food such as larvae. Fish species such as the tuskfish have been observed to dig up clams and to break them open by throwing them against a rock. Similar behavior has been observed among varieties of wrasse.

Tool use by humans had a similarly modest start. The stone age was marked by humans making tools of sharpened stone. These tools were used for hunting, crafting tools such as wooden tools, building structures, and warfare, among other uses. As human tool crafting and usage progressed, tools were made of bronze (an alloy of copper and tin), and later iron. Humans discovered that by adding further materials to iron to make steel, stronger, sharper, and more durable, tools could be made. These “new” tools enabled finer craftsmanship for the production of furniture, houses, bridges, and so on. The new steel tools were also better suited to warfare. As time progressed, humans created power sources to assist in manufacturing their tools. Wheels driven by wind or water were replaced by engines powered by steam, fossil fuels, and nuclear materials.

Electronic tools, including computers and software, have refashioned how humans live, work, and play. These tools perform calculations in mere tiny fractions of seconds that once required minutes or even hours. Modern machines automate manufacturing production that was once performed by legions of manual laborers. Food production today is on a scale that looks nothing like the family farms of old. Computer and telecommunication networks have grown in both capacity and complexity, allowing workers to collaborate from anywhere in the world. Teams of workers worldwide can share workloads, enabling complex efforts to be addressed by workers in all time zones. Human use of computers, mobile phones, tablets, pads, and so on has grown at a spectacular rate. Companies and industries, large and small, depend upon digital networks, computers, and human workers interconnected by electronic links. Today, many humans spend equal or more time interacting with their mobile devices than with other humans. As our use of technology continues to grow, the relationship between humans and machines, both physical and digital, continuously evolves.

### SUMMARY

Disclosed techniques enable context-aware gestures with an oral sensor interface. Users typically interact with processing devices by typing, clicking, touching, pressing, swiping, and employing other familiar input/output techniques. However, for some users and in some situations, these device manipulation techniques cannot be used due to impairments associated with a user. The impairments can include visual impairments, motor impairments, and cognitive impairments that prevent the user from providing input, receiving output, and so on. Other impairments that can be associated with a user include “situational impairments.” These latter impairments inhibit or prevent access to or usage of the input/output techniques due to a situation in which the user is trying to interact with the processing device. The situations can include lighting that is too bright or too dark, noisy environments, locations where speaking is not permitted, etc. The situation impairments can also be associated with those situations in which the user is using their eyes, ears, hands, voice, etc. for another activity, and therefore cannot access the usual input/output techniques. Any of these impairments render interacting with processing devices using standard input/output techniques difficult or impossible.

Data manipulation using context-aware gestures with an oral sensor interface is disclosed. An oral interface is accessed. The oral sensor interface includes a tongue position sensor (TPS). The TPS detects a tongue movement of the user. The oral sensor interface includes an inertial measurement unit (IMU). The IMU detects one or more head movements of the user. The oral sensor interface includes a barometric pressure transducer. The barometric pressure transducer recognizes the activation gesture. The oral sensor interface enables wireless connectivity to an operating system running on one or more processors. The oral sensor interface enables, with the operating system, oral mouse function control. The oral sensor interface is inserted into a user’s oral cavity. An activation gesture by a user is sensed by the oral sensor interface. The activation gesture selects, within the operating system, a mouse control mode within one or more mouse control modes.

A processor-implemented method for data manipulation is disclosed comprising: accessing an oral sensor interface, wherein the oral sensor interface enables wireless connectivity to an operating system running on one or more processors, and wherein the oral sensor interface enables, with the operating system, oral mouse function control; inserting the oral sensor interface into a user’s oral cavity; and sensing, by the oral sensor interface, an activation gesture by a user, wherein the activation gesture selects, within the operating system, a mouse control mode within one or more mouse control modes. In embodiments, the oral sensor interface includes a tongue position sensor (TPS). Embodiments include detecting, by the TPS, a tongue movement of the user. In embodiments, the oral sensor interface includes an inertial measurement unit (IMU). Embodiments include detecting, by the IMU, one or more head movements of the user. In embodiments, the oral sensor interface includes a barometric pressure transducer. Embodiments include detecting, by the barometric pressure transducer, a multi-value state of air pressure within the user’s oral cavity. The detecting accomplished by the sensors associated with the tongue position sensor is mapped to a plurality of oral gestures. The oral gestures are used for mouse control. The oral gestures can also be used for control of a mobility device such as a wheelchair.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow diagram for context-aware gestures with an oral sensor interface.

FIG. 2 is a flow diagram for selecting mouse control modes.

FIG. 3 is an infographic for context-aware gestures with an oral sensor interface.

FIG. 4 illustrates an oral sensing interface.

FIG. 5 is an example apparatus for context-aware gestures with an oral sensor interface.

FIG. 6 is an example of controlling modes.

FIG. 7 in an example table of mouse control modes.

FIG. 8 is a system diagram for data manipulation using context-aware gestures with an oral sensor interface.

### DETAILED DESCRIPTION

This disclosure provides techniques for intraoral data manipulation using context-aware gestures with an oral sensor interface. Users employ a diversity of techniques to interact with processors. The processors can include personal electronic devices such as smartphones, smartwatches, tablets, and personal digital assistants (PDAs); laptop and desktop computers; servers, whether local or cloud-based; and so on. The techniques by which users interact with processors can be based on typing on a keyboard, moving and clicking a mouse, swiping and tapping or pressing a trackpad, speaking into a microphone, reading contents rendered on a display, and so on. However, use of these typical input/output techniques for user-processor interaction is not always possible. One or more impairments can be associated with a particular user. Motor impairments can prevent a user from typing and controlling a mouse or trackpad, visual impairments can prevent a user from reading a display, and so on. Other impairments can include situational impairments. A situational impairment, which can impede or prevent a user from employing common input/output techniques, is based on a user’s particular situation. A situational impairment can include a bright light environment in which a user cannot read a display, a low light situation preventing accurate or any use of a keyboard, and the like. Situational impairments can also be based on circumstances of the user for which speaking violates laws, rules or social mores; accessing a screen is deemed rude and socially unacceptable; and the like. Further situations can include the user being engaged in another activity such as machine operation or surgery. In these latter cases, the user is unable to access a keyboard, mouse, or trackpad because the user’s hands are otherwise occupied.

Oftentimes people find themselves in situations where they have to multitask in order to address all that is asked of them. Tasks such as engaging in a video call while opening a file or initiating a computer search for an answer to a question that arose during the call have become routine. Viewing, talking, typing, mousing, swiping, and similar activities converge to enable and enhance the user experience. There are times, however, when overt interaction by a user with a computing or communication device is socially unacceptable, impossible, dangerous, or even illegal. In such situations, people are engaging in activities that require them to interact with processors while they are performing other tasks. The tasks can engage the user’s hands, thus preventing the user from engaging with common input/output devices associated with various computing and personal electronic devices. In these cases, disclosed intraoral data manipulation techniques can provide a “third hand” for a user. In other instances, the user may have one or more physical challenges or limitations which prevent human-machine interactions using the common input/output devices. These multifaceted requirements of the divergent tasks can include accessing a service manual while repairing or maintaining a piece of equipment, reading design specifications while operating a machine, or even using augmented reality while performing surgery. In addition, many situations exist in which a person cannot use conventional input/output devices or techniques. These latter situations can include low light conditions, limited access to input/output devices, or even covert activities.

Wireless connectivity is enabled by an oral sensor interface to an operating system running on one or more processors. The wireless connectivity can be based on wireless communications standards such as WiFi, Bluetooth, Zigbee, near field communications (NFC), cellular, and so on. The wireless connectivity can provide communications over distances ranging from a few millimeters, to centimeters, to meters, to kilometers, and so on. The oral sensor interface can include a tongue position sensor (TPS). The tongue position sensor can be located above the tongue, below the tongue, to the sides of the mouth, etc. The oral sensor interface can include an inertial measurement unit (IMU). The IMU can be used to detect the position, rotation, acceleration, etc., of the user’s head. The IMU can detect head gestures such as nods, shakes, tilts, and so on. The oral sensor interface can include a barometric pressure transducer. The barometric pressure transducer can detect barometric pressure within the user’s oral cavity, increases in barometric pressure, decreases in barometric pressure, rate of change of barometric pressure, duration of barometric pressure change, etc. The barometric pressure transducer can detect changes in air pressure within a closed space such as an oral cavity. The barometric pressure transducer can detect pressures along a continuum of pressures, that is, not just at specific pressures. However, the barometric pressure transducer can be used to detect a multi-value state of air pressure within the oral cavity containing the interface. The multi-value state includes ambient barometric pressure, increased barometric pressure due to exhaling into a closed oral cavity, and/or decreased barometric pressure due to inhaling from a closed oral cavity. The multi-value state collected from the barometric pressure transducer modifies the output of the TPS and the IMU. The oral sensor interface senses an activation gesture by a user. The activation gesture can be based on tongue movement, head position and movement, barometric pressure within the user’s oral cavity, and so on.

Data manipulation is enabled by an operating system running on one or more processors. The data manipulation is based on an activation gesture performed by the user. The activation gesture selects, within the operating system, a mouse control mode within one or more mouse control modes. The mouse control modes can include a tongue mode, a head mode, a speech mode, or another mode. In addition, the activation gesture itself can, for example, pull up a contextual menu, similar to the right click menu, but the interaction is through TPS gestures rather than point and click. The contextual menu can be displayed on a compute device, a mobile device, and so on. Any number of functions can be selected through the menus such as voice transcription, a keyboard function, a switching of devices to which the TPS is coupled, one or more shortcuts to functions, playing one or more media files, enabling a voice control. Thus, within the contextual menu, any touch, swipe sip, press, etc. can trigger any mouse button, scroll, keyboard input media control, etc. The menu can provide an option to return to the original mouse control mode.

An activation gesture can map to a different mouse control operation based a mouse control mode. The data manipulation can include opening and closing files, selecting menu items, data entry, etc. The data manipulation can be based on mouse and trackpad operations such as scrolling, swiping, clicking, double clicking, right clicking, and the like. The oral sensor interface can include a TPS, an IMU, and a barometric pressure transducer. The oral sensor interface can include one or more additional inertial measurement units. The inertial measurement unit and the at least one additional inertial measurement unit can be attached to nonadjacent locations along the oral sensor interface. The nonadjacent locations can include the left and right sides of the mouth, upper and lower jaw, and the like. The enabling data manipulation is augmented by the at least one additional inertial measurement unit. Feedback can be provided to a user of the oral sensor interface. The feedback can include haptic feedback generated by a haptic device, audio feedback generated by a sound generating device, etc.

In disclosed techniques, data manipulation uses context-aware gestures with an oral sensor interface. The oral sensor interface can enable wireless connectivity with an operating system running on one or more processors. Wireless connectivity is provided between a processor and a wireless transceiver. The wireless transceiver is embedded in an oral sensing interface. The oral sensor interface includes a tongue position sensor (TPS). The oral sensing interface detects tongue gestures where the tongue gestures can include a plurality of activation gestures. The oral sensor interface includes an inertial measurement unit (IMU). The IMU detects one or more head movements of the user. The oral sensor interface includes a barometric pressure transducer. The barometric pressure transducer detects a multi-value state of air pressure within the user’s oral cavity. The barometric pressure transducer detects air pressure within an oral cavity containing the interface. The barometric sensor input air pressure is supplied by a barometric sensor diaphragm which can be integrated with the TPS. Data manipulation is enabled on an operating system running on one or more processors. The one or more processors associated with the oral sensor interface can sense an activation gesture by the user. The sensor data is transferred to the operating system based on the wireless connectivity. Feedback in the form of both haptic feedback and audio feedback can be provided to the oral sensor interface user.

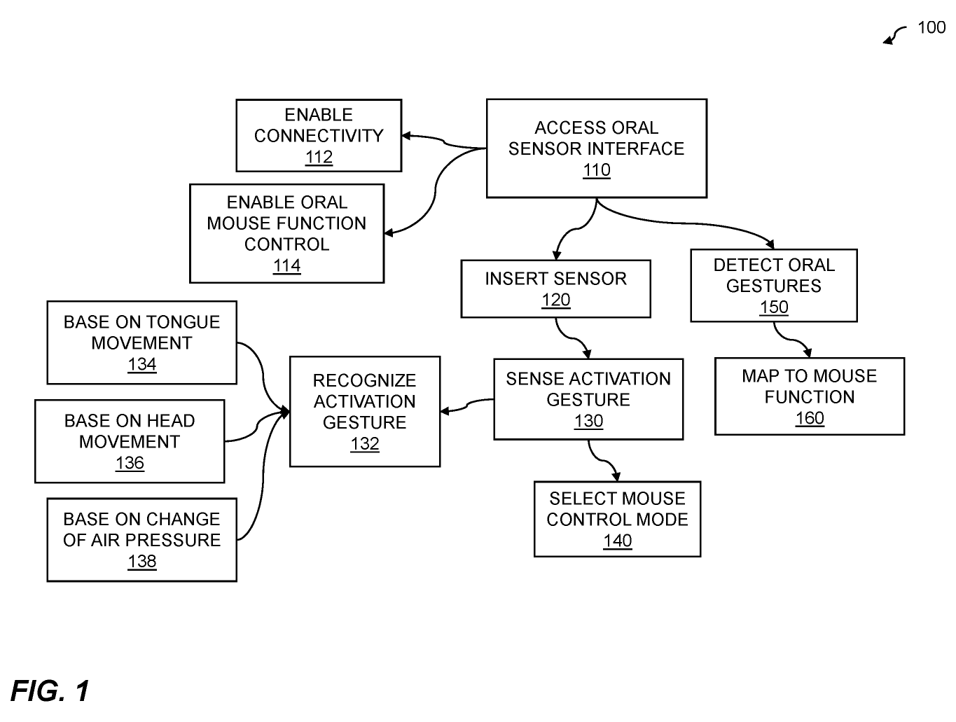

FIG. 1 is a flow diagram for context-aware gestures with an oral sensor interface. Data manipulation is accomplished using context-aware gestures that are detected based on sensor data obtained from a plurality of sensors. The sensing is implemented for processor-based data manipulation using an oral sensor interface. Wireless connectivity is enabled to an operating system running on one or more processors. The wireless connectivity can be accomplished using a wireless transceiver. The wireless transceiver can be embedded in an oral sensing interface. The oral sensing interface can include a tongue position sensor (TPS). The TPS detects tongue movement of the user. The detecting includes three-dimensional movements of the tongue of the user. The oral sensor interface can include an inertial measurement unit (IMU). The IMU detects one or more head movements of the user. The oral sensor interface can include a barometric pressure transducer. The barometric pressure transducer detects a multi-value state of air pressure within the user’s oral cavity. The barometric sensor input air pressure can be supplied by a barometric pressure transducer integrated with the TPS. The oral sensor interface senses an activation gesture by a user. The activation gesture selects, within the operating system, a mouse control mode within one or more mouse control modes. The mouse control modes can include a tongue mode, a head mode, a speech mode, or another mode. A user may operate various sensors to enable data manipulation in a processor while the user is experiencing situational impairment. Situational impairment can refer to a user’s inability to interact with a computer, processor, and so on due to their situation.

The flow 100 includes accessing 110 an oral sensor interface. An oral sensor interface can be worn by a user, placed within the user’s oral cavity, and so on. In embodiments, the oral sensing interface can include a retainer or “bite plate.” The retainer can be custom fitted to the user in order to enable both proper fit of the sensor interface and comfort of the user. A battery can be coupled to the wireless transceiver. The battery can include a replaceable battery, a rechargeable battery, and so on. In a usage example, the battery can be charged wirelessly in a user’s oral cavity. In another usage example, the battery can be replaced, charged, and/or augmented with a piezoelectric layer included in the oral sensor interface. Natural movements of the jaw can enable electrical energy production during normal jaw movements such as chewing, talking, and the like. The flow 100 includes enabling wireless connectivity 112 to an operating system running on one or more processors. The wireless connectivity can be based on communications standards, preferred protocols, low power techniques, and so on. The wireless connectivity can be based on the 802.11 family or Wi-Fi, Bluetooth, Zigbee, and so on. The wireless connectivity can be based on near field communication (NFC). The wireless connectivity can be based on near field magnetic induction (NFMI). The wireless connectivity can be provided as part of a wireless personal area network (WPAN). The wireless connectivity can be enabled by using a wireless transceiver to implement the desired wireless connectivity. The wireless transceiver can be embedded in an oral sensor interface. The operating system can include a standard operating system such as UNIX, Windows, macOS, and so on. The operating system can be associated with a handheld or portable device. The latter operating system can include Android, iOS, iPadOS, etc. The one or more processors can include a handheld device, a portable device, a tablet, a computer, a server, etc.

In the flow 100, the oral sensor interface enables, with the operating system, oral mouse function control 114. Mouse function control can include moving a cursor, clicking, and so on. The oral mouse function control can include mouse function detection, such as mouse movement, mouse swipes, mouse clicks, mouse double-clicks, mouse wheel control, etc. Further mouse functions, such as secondary functions, can be based on an amount of pressure exerted by the user’s tongue, the user’s breath, the user’s head position, and so on. In a usage example, the data manipulation can be targeted for one or more Internet of Things (IoT) devices. Such data manipulation can potentially enable control of any internet-connected device using remote augmented sensing, and in particular, oral remote augmented sensing.

The flow 100 includes inserting the oral sensor interface 120 into a user’s oral cavity. The user’s oral cavity includes the user’s mouth. The oral sensor interface can use one or more sensors, where data collected from the one or more sensors can be used for mouse control. The oral sensor interface can be applied to other control applications such as control of a mobility vehicle. The mobility vehicle can include a wheelchair. Thus, the oral sensor interface can be coupled to a variety of sensors. In embodiments, the oral sensor interface includes a tongue position sensor (TPS). The tongue position sensor can be positioned on the oral sensor interface such that it can be easily accessible to the tongue. The TPS can be placed below the tongue of the user, such as when the retainer is worn on the lower teeth. An alternative location for the TPS can be above the tongue, such as when the retainer is worn on the upper teeth. The TPS can include capacitive sensors, electrodes, pressure sensors, optical sensors, ultrasonic sensors, etc. Tongue input data can be detected by the TPS and can augment, control, or modify data collected by, or being processed by, a processor, preprocessor, etc. The tongue input data can be sensed based on tongue position, tongue portion, and tongue pressure. Embodiments include detecting, by the TPS, a tongue movement of the user. The tongue movement can include tongue movement direction, tongue movement speed, tongue movement acceleration, and so on. In embodiments, the detecting includes three-dimensional movements of the tongue of the user. A capacitive-based sensor can trace movements of a user’s tongue across the surface of the sensor including two-dimensional (2D) movement, tongue gestures, tongue taps, tongue presses, tongue movement acceleration, and so on. The oral sensor interface can control a two dimensional velocity of a cursor on a screen coupled to a computer, mobile device, etc. The speed of the cursor can be controlled by a distance of the tongue, on the TPS, from its center. The further from the center of the TPS is touched, the faster the cursor speed can be displayed. The TPS can be directionally sensitive. For example, a touch by the tongue of a user on the right side of the TPS can move the cursor to the right at a speed indicated by the distance of the touch from the center of the TPS. The tongue position sensor detects tongue pressure independently of other sensors, including a barometric pressure sensor (discussed below). Other TPS technology may be able to provide three-dimensional tongue movement sensing.

In embodiments, the oral sensor interface includes an inertial measurement unit (IMU). More than one IMU can be included in the sensor interface. The IMU can measure a variety of parameters associated with a body in motion. The IMU can sense data associated with one or more of acceleration, angular velocity, magnetic field, and barometric pressure. The IMU can measure head position, head tilt angle, head rotation, head rotation velocity, head rotation acceleration, etc. An additional inertial measurement unit can be attached to an additional location of the oral sensor interface (retainer), such as a nonadjacent location to the first IMU. Embodiments include detecting, by the IMU, one or more head movements of the user. The head movements of the user can include a head tilt, a head rotation up or down, a head rotation left or right, and so on. The head movements can include a series of movements such as a rotation left to right to center (e.g., a nod indicating “no”), a rotation up to down to center (e.g., a not indicating “yes”), and the like.

In embodiments, the oral sensor interface includes a barometric pressure transducer. The barometric pressure transducer can be used to detect ambient barometric pressure, changes in barometric pressure such as pressure increases or pressure decreases, and so on. The barometric pressure transducer can detect various states of pressure changes. The barometric pressure transducer can detect along a continuum of pressures to provide a virtual analog type of input to a processor. The barometric pressure transducer can perform these detections based on regions, or states, of pressure. Embodiments include detecting, by the barometric pressure transducer, a multi-value state of air pressure within the user’s oral cavity. For example, a tri-value state of air pressure can be detected within an oral cavity containing the oral sensor interface. The tri-value state can include a number, a code, a percentage, text, and the like. The tri-value state can be sensed from a continuum of pressures and can include ambient barometric pressure, increased barometric pressure due to exhaling into a closed oral cavity, and decreased barometric pressure due to inhaling from a closed oral cavity. The barometric pressure transducer can detect a continuous range of pressures, such as a plurality of pressures along an analog or continuum of possible pressures for sensing. In a usage example, an output of the barometric sensor can be used in the enabling data manipulation, either in conjunction with outputs of other sensors or by itself, and it can modify the output of other sensors when included in the oral sensing interface. The tri-value state can include ambient barometric pressure, increased barometric pressure due to exhaling into a closed oral cavity, and decreased barometric pressure due to inhaling from a closed oral cavity. The integrated barometric pressure transducer can be encapsulated with the TPS such that a flexible printed circuit capacitive TPS can be encapsulated over a sealed region under the TPS that provides a diaphragm structure to feed the barometric sensor. Thus, air movement in the encapsulated TPS/barometer diaphragm can be directed to a convenient location distinct from the TPS, such as a nearby electronics circuit board, which can house the actual barometric pressure transducer itself, along with other electronics, such as a processor or preprocessor, wireless connectivity device, ambient sensors, and so on.

The barometric pressure transducer, that is, the barometric pressure transducer proper that is connected to the integrated and encapsulated barometric diaphragm, can be used to detect tongue gestures (movement, taps, presses, etc.) either in conjunction with the TPS or in a standalone manner. For example, a capacitance-based TPS might require a very thin encapsulation in order to maintain sensitivity in a typically wet oral cavity environment. Force pressure sensors can be added. The force pressure sensors can be below the TPS. However, the added thickness can interfere with a user’s speech. Thus, the force pressure sensors can be in various locations so that they do not unnecessarily add to the thickness of the TPS. In embodiments, the integrated barometric sensor can augment the TPS to provide a more complete indication of tongue gestures. The thin encapsulation also enables less interference with a user’s speech, and in fact, thinness for the entire oral sensing interface is desirable for that reason.

The barometric sensor can detect a penta-value state. The penta-value state includes five different states that can be detected by the barometric sensor: ambient (nothing or no change), modestly higher pressure (a soft blow of breath into a closed mouth), high pressure (a hard blow of breath into a closed mouth), modestly lower pressure (a soft draw of breath into a closed mouth), and low pressure (a hard draw of breath into a closed mouth). The penta-value state can represent five different values, options, numbers, code words, alphanumeric symbols, punctuation marks, and so on. The barometric sensor can detect a continuum of pressures. A penta-value mode, a tri-value mode, a continuum mode, or another mode can be activated by a mode selection, controlled either by an external processor or an in-mouth processor. The tongue position sensor and the barometric pressure sensor enable oral mouse function detection. Oral mouse function allows mouse, trackball, or trackpad functionality using only a user’s oral cavity faculties.

The flow 100 includes sensing 130, by the oral sensor interface, an activation gesture by a user. An activation can include a tongue gesture, a head gesture, a barometric pressure “gesture,” and so on. In embodiments, the sensing is based on a machine learning model. The machine learning model can learn to identify activation gestures in general and activation gestures as presented by an individual user. For example, various users can generate different levels of pressure as detected by the barometric pressure transducer. The machine learning model can detect an appropriate activation gesture for various users. The gesture does not need to be an oral gesture. The gesture can comprise an in-body activation gesture. For example, the gesture can be a foot tap, a clap, a finger tap, a wink, brain electrical activity, and so on. Additional sensors such as microphones, cameras, electroencephalography (EEG) sensors, magnetoencephalography (MEG) sensors, and so on can be provided, if needed, to capture other non-oral activation gestures. The machine learning model can be used to recognize the in-body activation gesture.

The gesture can be used to control a computer interface device such as a mouse device. The sensing an activation gesture can be based on sensing by the TPS, the IMU, the barometric pressure transducer, and other sensors that can be coupled to the oral sensor interface. In the flow 100, the activation gesture can be recognized 132. Since a variety of activation gestures can be included to activate one or more mouse control functions, an activation gesture from a plurality of activation gestures can be recognized. In a usage example, the activation gestures can activate a mouse click, a mouse double click, a swipe, etc.

In the flow 100, the sensing includes recognizing, by the TPS, the activation gesture, wherein the activation gesture is based on the tongue movement 134. The tongue movement can include a three-dimensional movement. More than one oral gesture can be detected (discussed below). In the flow 100, the sensing includes recognizing, by the IMU, the activation gesture, wherein the activation gesture is based on the head movement 136. Discussed previously and throughout, the head movement can include head position or orientation, head tilt, head rotation, head rotation velocity, head rotation acceleration, and the like. In the flow 100, the sensing includes recognizing, by the barometric pressure transducer, the activation gesture, wherein the activation gesture is based on a change of the multi-value state of air pressure 138 within the user’s oral cavity. The state of air pressure within the user’s oral cavity can include a pressure value, a change in pressure value such as an increase in pressure or a decrease in pressure, a duration of a pressure change, and the like. The barometric pressure transducer can be used to detect many various types of “gestures” by the user such as sucking, sipping, or blowing; spoken “gestures” such as spoken words, spoken phrases, and nonlanguage utterances; and so on. The speech gestures can be detected by the biometric pressure transducer, a microphone, another device, or a combination of devices. The various sensors associated with the oral sensor interface can be used singly, in combination, and so on to recognize an activation gesture.

In the flow 100, the activation gesture selects 140, within the operating system, a mouse control mode within one or more mouse control modes. The mouse functionality of the mouse control mode can include typical mouse functions as well as functionality typical of other input devices such as keyboards, trackpoints, mousepads, joysticks, game controllers, and so on. The mouse functionality can include the functionality of various other human interface devices (HIDs) as well. The selecting can be based on sensor data from a single sensor, sensor data from more than one sensor, and so on. The selecting can select a mouse control mode based on a gesture, the sensor data used to send an activation gesture, and the like. In a usage example, the activation gesture can be received by the TPS. More than one mouse control mode can be associated with the operating systems running on the one or more processors. In embodiments, the one or more mouse control modes include a tongue mode. The tongue mode can map the activation gesture to a first subset of mouse control functions. In embodiments, the one or more mouse control modes include a head mode. The tongue mode can map the activation gesture to a second subset of mouse control functions. In embodiments, the one or more mouse control modes include a speech mode. The speech mode can map the activation gesture to a third subset of mouse control functions. In a usage example, a user performs a directional tongue movement. This one gesture can map to different mouse controls based on the control mode. The directional tongue movement can map to a cursor control function in tongue mode, a scrolling function in head mode, and a menu selection function in speech mode. The tongue touch position gesture automatically controls the 2D velocity of the cursor.

Instead of selecting a mouse control mode, the activation gesture can invoke a contextual menu which can provide visual feedback to a user. The contextual menu can be displayed on a compute device such as a computer, laptop, tablet, smart phone, and so on. Any number of functions can be selected through the menu such as voice transcription, a keyboard function (e.g., the TPS can sense a tap on a keyboard based on a position of the tongue on the sensor), a switching of devices to which the TPS is coupled, one or more shortcuts to functions, playing one or more media files, enabling voice control of the computer device, returning to the original mouse control mode, and so on. Any number of functions can be included in the menu. Selection of an option in the menu can be made via the two dimensional velocity control function of the TPS as described earlier. Thus, the velocity vector that represents the tongue’s movement, position, etc. can now select among a discrete set of actions such as described above.

In the flow 100, the oral sensor interface detects 150 a plurality of oral gestures. The detecting a plurality of oral gestures can be based on a compound activation gesture. A compound activation gesture can include two or more gestures being performed substantially simultaneously or within a time delta. In a usage example, a compound activation gesture could include the user contacting their tongue to the TPS while nodding a “no” gesture. The plurality of oral gestures can include a sequence of two or more activation gestures. In a usage example, the user could execute a tongue movement, a head rotation, and a speech utterance in an order. The flow 100 further includes mapping the plurality of oral gestures to a mouse function 160, wherein the mapping is based on the mouse control mode. The mouse function can include cursor control scrolling, a menu selection, an idle/stop motion function, a stop cursor function, a left-click, a punctuation menu, and so on. Recall that the mouse function to which the activation gesture is mapped is dependent on the one or more control modes.

The data manipulation can be used to accommodate situational impairment experienced by a person. The impairments can include visual, motor, and cognitive impairments. In a usage example, the sensor-based data manipulation enablement can be used to accommodate severe motor impairment in a person, which can limit or prevent the person from accessing a processor using conventional devices such as keyboards, mice, trackpads, monitors, and so on. The impairment can include upper limb motor control impairment. Different from visual, motor, or cognitive impairments, situational impairments occur when users are not able to use processors and devices because of a situation in which they find themselves. Such situations can occur due to ambient conditions such as too much light to read a screen or too little light to use a keyboard or trackpad; too loud an environment to hear audio feedback from the processor or computing device; too little ambient noise where speaking would disturb others such as in a library or at a movie; etc. Other causes of situational impairment can include an inability to use standard input/output devices when it is socially unacceptable; when a user’s hands, eyes, and voice are otherwise occupied; and when covert interaction with a processor is required. Some situational impairments can be associated with a particular user due to their physiological, mental, or emotional situation. For example, motor impairments can prevent a user from typing and controlling a mouse or trackpad; visual impairments can prevent a user from reading a display; and so on. The situational impairment accommodation can enable control of a joystick functionality without having hand motor skills to control it traditionally. Examples can include operating a wheelchair or controlling another vehicle type. The situational impairment can include operating a machine. The machine can generate too much noise to hear audio feedback or to provide voice commands, can require two hands to operate, and the like. The situational impairment can include performing a surgical procedure.

In a usage example, feedback can be provided to a user of the interface. The feedback that is provided to the user of the interface can include results of data manipulation enabled in a processor, alerts, warnings, codes, and so on. In a usage example, the feedback can include haptic feedback generated by a haptic device attached to the oral sensor interface. The haptic feedback can include vibration, pressure, a tingling sensation, and the like. In another usage example, the feedback can include audio feedback generated by a sound generating device attached to the interface. The sound generating device can include a speaker, a transducer, and so on. The sound generating device can use jawbone structure for audio propagation. Other feedback forms, such as electrical stimulation, are possible and can be used with haptic and/or audio feedback or by themselves.

Continuing with feedback, the feedback can originate in the processor and can be transmitted to the interface using the wireless connectivity. The wireless connectivity can be based on the various wireless techniques discussed throughout. In other embodiments, the feedback can originate in the processor and can be based on output previously received by the processor over a connection using the wireless connectivity. The output previously received by the processor can include TPS data, IMU data, barometric pressure transducer data, data from other sensors that can be coupled to the interface, and the like. The feedback can be based on an action taken by the user. In a usage example, the feedback can be responsive to active oral manipulation by a user of the interface. The active oral manipulation can include the user moving their tongue to move the cursor, click, etc. The active oral manipulation can accomplish data manipulation in the processor. The active oral manipulation can access files for input and output, operate applications, and so on. The feedback can be responsive to passive monitoring of a person using the interface. The passive monitoring can include monitoring the user’s health, habits, activities, etc. In a usage example, the feedback can be used to control bruxism in the user. Bruxism, or “teeth grinding,” can be quite harmful to the teeth. In another usage example, the feedback can be used to control sleep apnea in the user. Sleep apnea can result from the airway closing off while sleeping. Sleep apnea can manifest as loud snoring, gasping, and so on, and has many undesirable side effects which can include headaches, depression, stroke, or heart failure.

Various steps in the flow 100 may be changed in order, repeated, omitted, or the like without departing from the disclosed concepts. Various embodiments of the flow 100 can be included in a computer program product embodied in a non-transitory computer readable medium that includes code executable by one or more processors.

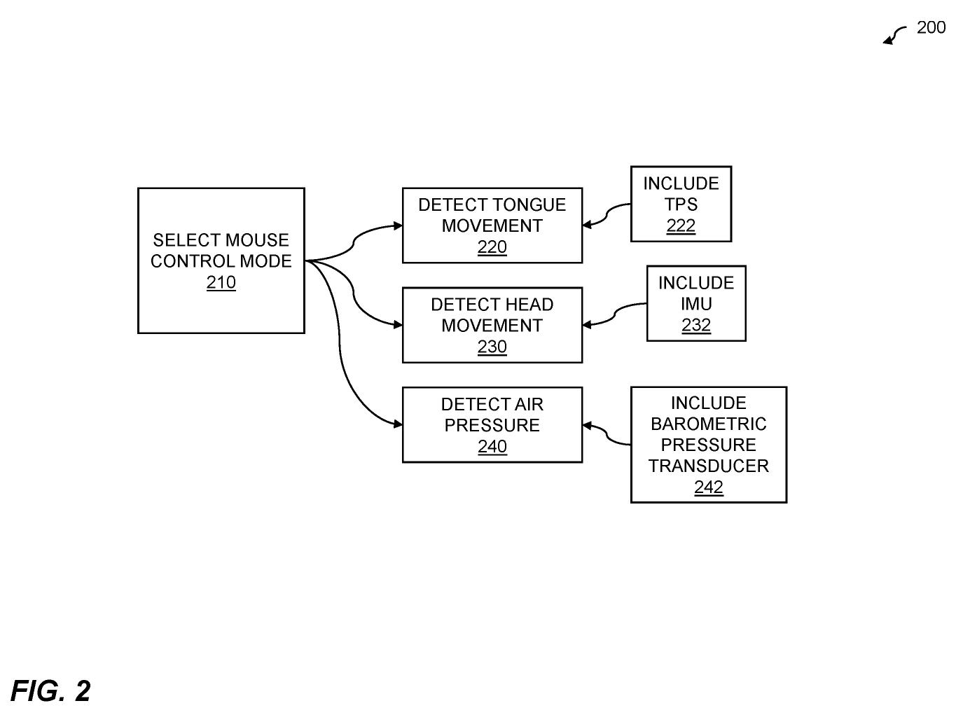

FIG. 2 is a flow diagram for selecting mouse control modes. Sensors embedded in an oral sensor interface capture data from a user interacting with the oral sensor interface. The selecting mouse control modes is accomplished using explicit control and implicit control. The explicit control can be selected by a user. The implicit control can be based on an agent, where the agent can select a mode based on an application that the user is operating. Sensors within an oral sensor interface can enable data manipulation using context-aware gestures. The oral sensor interface can support a user-processor interface that can be applied by the user to enable data manipulation within a processor. The oral sensor interface can further enable operation of a mobility device such as a wheelchair. The data manipulation can include opening, operating, and closing applications; selecting data files; writing data files; and so on. The oral sensor interface can enable the user to operate the processor, execute programs, etc. when the use of “standard” interfaces such as keyboards, mice, trackpads, microphones, headphones, etc., is impossible, impractical, socially unacceptable, and so on. These operating conditions, such as noisy environments, quiet environments (for instance, a library or a courtroom), overly bright or dim environments, etc., contribute to “situational impairment.” Situational impairment refers to a situation in which accessing processors, computers, and the like, is difficult, socially unacceptable, imprudent, etc.

The flow 200 includes selecting 210, within an operating system, a mouse control mode within one or more mouse control modes. An activation gesture by a user is sensed by the oral sensor interface. The activation gesture selects a mouse mode. The activation gesture can be accomplished by the user using their tongue to contact the oral sensor interface. The contact can be based on a location of contact, a movement, a pressure, and so on. In a usage example, the activation gesture can include a direction of a tongue movement such as left or right, a full tongue press, a partial tongue press, a partial tongue press and hold, a partial long tongue press, suction or a sip, suction and hold, long suction, etc. The sensing an activation gesture can be based on detecting tongue movement, detecting head movement, or detecting air pressure, as discussed below.

The flow 200 includes detecting a tongue movement 220 of the user. The tongue movement can include a full tongue pressure, a partial tongue pressure, a tongue movement left or right, a tongue movement forward or back, and so on. In embodiments, the detecting includes three-dimensional movements of the tongue of the user. The tongue movement can include a tongue movement and a tongue press. In embodiments, the oral sensor interface includes a tongue position sensor (TPS). In the flow 200, the detecting a tongue movement is accomplished by a tongue position sensor (TPS) 222. Recall that the oral sensor interface can include a prosthetic device such as a retainer. The retainer can be fitted to the user so that the retainer can be worn comfortably by the user. The TPS can be attached to the oral sensor interface. The TPS can be electrically coupled to a wireless transceiver unit which can enable communication to an operating system running on one or more processors. The detecting the tongue movement can be accomplished by a processor such as a preprocessor attached to the retainer.

The flow 200 includes detecting one or more head movements 230 of the user. The one or more head movements can include a head position before a head move and a head position after a head move, a head tilt, a head rotation, and so on. The head rotation can include a rotation to the left or a rotation to the right, a rotation up or a rotation down, etc. The head rotation can include a “nod” such as a left-right nod (e.g., “no”), an up-down rotation (e.g., “yes”), and the like. In the flow 200, the oral sensor interface includes an inertial measurement unit (IMU) 232. The IMU can measure head position, head tilt angle, head rotation, head rotation velocity, head rotation acceleration, etc.

In the flow 200, the oral sensor interface includes a barometric pressure transducer 242. The barometric pressure transducer can include a barometric pressure-sensing diaphragm. The barometric pressure transducer can be integrated with TPS. The integration can comprise an encapsulation of the TPS along with a narrow hollow space under all or part of the TPS that is pressure sensitive and can shunt encapsulated air, and thereby pressure, from the region under the TPS to a nearby barometric sensor proper. Embodiments include detecting, by the barometric pressure transducer, a multi-value state of air pressure 240 within the user’s oral cavity. The multi-value state of air pressure within the user’s oral cavity can include an increased pressure state, a decreased pressure state, a changing pressure state, a static pressure state, and so on.

Various steps in the flow 200 may be changed in order, repeated, omitted, or the like without departing from the disclosed concepts. Various embodiments of the flow 200 can be included in a computer program product embodied in a non-transitory computer readable medium that includes code executable by one or more processors.

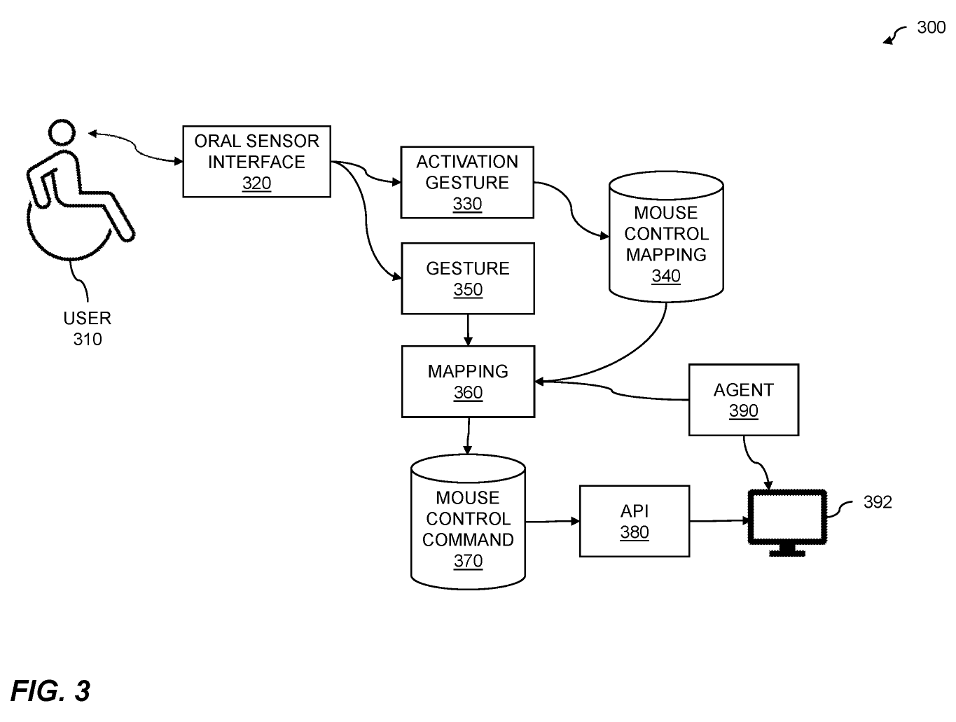

FIG. 3 is an infographic for context-aware gestures with an oral sensor interface. The oral sensor interface can include one or more sensors such as a tongue position sensor (TPS), an inertial measurement unit (IMU), a barometric pressure transducer, and so on. The oral sensor interface can use the sensors to sense an activation gesture by the user. An activation gesture can be mapped to a mouse function based on a selected mouse control mode. The mouse control mode can include a tongue mode, a head mode, a speech mode, or another mode. Thus, a given activation gesture can be mapped to a first mouse function based on a first mode, a second mouse function based on a second mode, and so on. Further, a context can be used to determine a mode, a set of configuration parameters used to set mouse operations, and so on. A context can be based on an application or app such as a word processing app, a game, and so on. Data manipulation is accomplished using context-aware gestures with an oral sensor interface. An oral sensor interface is accessed. The oral sensor interface enables wireless connectivity to an operating system running on one or more processors. The oral sensor interface enables, with the operating system, oral mouse function control. The oral sensor interface is inserted into a user’s oral cavity. An activation gesture by a user is sensed by the oral sensor interface. The activation gesture selects, within the operating system, a mouse control mode within one or more mouse control modes.

The infographic 300 includes a user 310. The user can include any user who desires to operate a device based on activation gestures. The device can include a handheld device, a tablet device, a computer, a server, a cloud server, and so on. The device can also include an assistive device such as a mobility device. In a usage example, the mobility device can include a wheelchair. The user can access an oral sensor interface 320. The oral sensor interface can enable control of a device such as a computer mouse, a mobility device, etc. The oral sensor interface can include a prosthetic device such as a retainer that can be worn in an oral cavity of the user. In embodiments, the oral sensor interface enables wireless connectivity to an operating system running on one or more processors, wherein the oral sensor interface enables, with the operating system, oral mouse function control. The oral mouse function control can include clicking, double clicking, selection, swiping, cursor control, and the like. The oral sensor interface can include one or more sensor devices. In embodiments, the oral sensor interface includes a tongue position sensor (TPS). The TPS can detect tongue contact, tongue pressure, tongue movement, and so on. In embodiments, the oral sensor interface includes an inertial measurement unit (IMU). The IMU can detect head position, head tilt, head rotation, and the like. In other embodiments, the oral sensor interface includes a barometric pressure transducer. The barometric pressure transducer can detect air pressure within the user’s oral cavity, changes in air pressure, etc.

The infographic 300 includes an activation gesture 330. The activation gesture can be sensed based on sensor data from one or more sensors. An activation gesture can be based on a sensor data from a tongue position sensor (TPS), an inertial measurement unit (IMU), a barometric pressure transducer, and so on. Other sensors can be associated with the oral sensor interface such as a microphone, an ambient condition sensor, biosensors, and the like. Embodiments include detecting, by the TPS, a tongue movement of the user. Discussed throughout, the tongue movement can include a movement direction, a movement velocity, etc. The movements can include three-dimensional movements. In embodiments, the sensing includes recognizing, by the TPS, the activation gesture, wherein the activation gesture is based on the tongue movement. In embodiments, the sensing includes recognizing, by the IMU, the activation gesture, wherein the activation gesture is based on the one or more head movements of the user.

The head movements can include a head tilt, a head rotation, a head nod, and so on. In embodiments, the sensing includes recognizing, by the barometric pressure transducer, the activation gesture, wherein the activation gesture is based on a change of the multi-value state of air pressure within the user’s oral cavity. The multi-value state of air pressure within the user’s oral cavity can be based on a sip of air, adding a portion of air to the oral cavity, removing a portion of air from the oral cavity, etc.

The infographic 300 includes mouse control mapping 340. Activation gestures can be mapped to one or more control actions for a device such as a computer mouse. The activation gestures can be mapped to other control actions for other devices such as mobility devices. Embodiments include mapping the plurality of oral gestures to a mouse function, wherein the mapping is based on the mouse control mode. A mouse control mode can include a mouse control mode within a plurality of mouse control modes. The mouse control modes can include a tongue mode, a head mode, a speech mode, and so on. Thus, an oral gesture can be mapped to more than one control operation based on a mouse control mode. In a usage example, a user of the oral sensor interface can move their tongue in a direction. The activation gesture represented by the tongue movement can map to cursor control in tongue mode, to scrolling in head mode, and to menu selection in speech mode.

The infographic 300 can include one or more gestures 350. The gestures can include activation gestures. The activation gestures can be based on tongue gestures, head gestures, speech gestures, and so on. The gestures can be detected by the oral sensor interface. Discussed previously and throughout, the tongue gestures can include a tongue direction; a full tongue press; a partial tongue press; a partial tongue press and hold; a partial tongue long press; suction (e.g., a sip); suction and hold; long suction; and so on. The head gestures can include head position; head tilt; head rotation; head rotation velocity; and the like. The speech gestures can include spoken words, spoken phrases, nonlanguage utterances, etc. The speech gestures can be detected by the biometric pressure transducer, a microphone, or another device.

The infographic 300 can include mapping 360. The mapping can include mapping a gesture to a mouse control command. The mouse control command can be based on a gesture such as oral gestures mapped to the mouse control command. Recall that the mouse function or control command is based on the mouse control mode discussed previously. The mapping can be based on explicit control and implicit control. Explicit control can include control selected by the user, control based on parameters settings for a given application, and so on. The implicit control (discussed below) can be based on a context. The context can include a user application, user preferences, and the like. The control can be automatically set by the implicit control. Note that explicit control can override implicit control.

The infographic 300 can include one or more mouse control commands 370. Discussed previously and throughout, the mouse control commands can include clicking, double clicking, swiping, scrolling, cursor control, and so on. The mouse control commands can be enabled by an application programming interface (API) 380. In embodiments, the enabling is based on one or more application programming interfaces (APIs). An API can map the mouse control command selected by the user’s activation gesture sensed by the oral sensor interface to a command, instruction, operation, macro, etc. that can execute the mouse control operation. The API can comprise a Bluetooth low energy human interface device (BLE HID) interface. The infographic 300 can include an agent 390. The agent can execute on a device such as a smartphone, a tablet, a computer, and so on. The agent can identify applications or apps being executed by the user and can determine a context for the execution. The agent can provide implicit control based on the application and the context. In a usage example, the user can be executing a word processing application. The mouse controls can enable cursor movement, menu item selections, insertion of text, deletion of text, and so on. In a second usage example, the user can be playing a video game. The mouse control can include rapid response to activation gestures such as changes in direction and speed, weapon selection, weapon operation, etc. The mouse control commands based on mapping of gestures to mouse control commands and the determinations by the agent can be executed on a device 392. The device can include a computing device, a mobility device, etc.

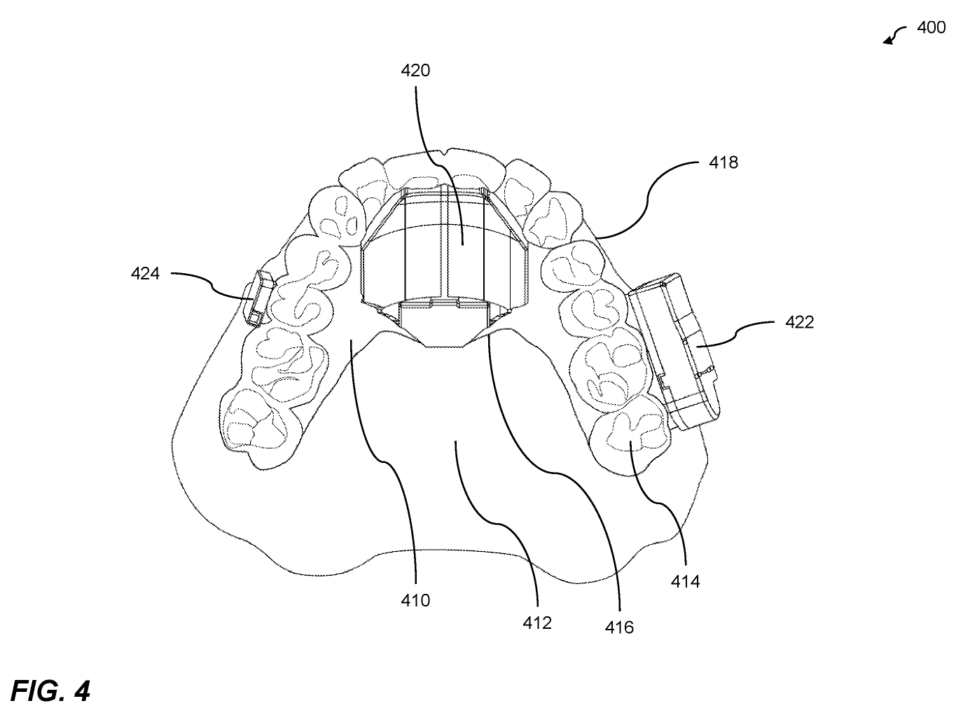

FIG. 4 illustrates an oral sensing interface. An oral sensing interface can accomplish data manipulation based on context-aware gestures. The context-aware gestures can be determined from sensor data collected by sensors associated with the sensing interface. In the illustration 400, the oral sensing interface can take the form of a dental retainer. One or more of tongue position sensors (TPSs), inertial measurement units (IMUs), barometric pressure transducers, other sensors, wireless transceivers, preprocessors, and so on can be attached to or along a physical structure, such as a retainer, for in-mouth use. The retainer can be used by placing the retainer into the oral cavity, or mouth, of the user. The retainer, as configured to operate as an oral sensing interface, can detect tongue pressure, tongue movement, head movement, barometric pressure, and so on. The interface, or retainer, enables data manipulation using context-aware gestures with an oral sensor interface. An oral sensor interface is accessed. The oral sensor interface enables wireless connectivity to an operating system running on one or more processors. The oral sensor interface enables, with the operating system, oral mouse function control. The oral sensor interface is inserted into a user’s oral cavity. An activation gesture by a user is sensed by the oral sensor interface. The activation gesture selects, within the operating system, a mouse control mode within one or more mouse control modes.

Discussed throughout, a tongue position sensor, one or more inertial measurement units, and other sensors can be coupled to a device that can be placed into an oral cavity (e.g., a mouth) of a user. In embodiments, the device can include a retainer 410. The retainer 410 shown is clear and covers a palate 412 replete with teeth, such as tooth 414. The retainer 410 is illustrated by boundary edge 416 (more toward the back of the mouth) and boundary edge 418 (more toward the front of the mouth). The retainer can include a customized retainer, where the customized retainer is fitted to a particular user to ensure proper fit and to enable comfort of the user. The retainer can be fitted to cover the upper teeth or the lower teeth. A retainer covering the upper teeth may be more comfortable to a user. A retainer covering the lower teeth can enable greater motion detection, due to the anatomical mobility and freedom of movement of the lower jaw vis-à-vis the upper jaw. A tongue position sensor (TPS) 420 can be attached to the retainer and can be electrically coupled to a wireless transceiver unit 422. The electrical connectivity can be provided by wire traces embedded in the retainer. The wire traces can comprise serpentine shapes to allow for retainer deformation without wire breakage. The electrical coupling can be accomplished wirelessly. The TPS can detect tongue gestures such as tongue movement, tongue position, tongue pressure, tongue movement speed, tongue movement acceleration, and so on. A barometric pressure transducer such as a barometric pressure-sensing diaphragm can be integrated with TPS 420. The integration can comprise an encapsulation of the TPS along with a narrow hollow space under all or part of the TPS that is pressure sensitive and can shunt encapsulated air, and thereby pressure, from the region under the TPS to a nearby barometric sensor proper. Note that for mouth roof-mounted oral interfaces, the relative position is reversed, and the barometric diaphragm is above the TPS, when viewed from the perspective of an upright user of the device. Thus, “above” and “below” are relative terms in this context, depending on the orientation of the TPS within the oral cavity.

Other sensors can be coupled to the retainer. An inertial measurement unit (IMU) can be included with and coupled to wireless transceiver unit 422. The IMU can be used to determine position, acceleration, and rotation of the jaw or head of the user. In embodiments, an additional inertial measurement unit 424 can be attached to a nonadjacent location of the interface (retainer) and coupled electrically to the wireless transceiver unit 422. The additional inertial measurement unit (424) can also be integrated with the wireless transceiver unit 422. The unit and the sensor can include further sensors such as barometric pressure sensors, biometric sensors, bone-conduction microphones, bone-conduction sound generation devices, and so on. Additional sensors can be attached to the retainer at other locations along the retainer. In embodiments, pressure sensors can be embedded in or along the surface of the retainer. The pressure sensors can be used to measure pressure such as pressure resulting from clenching teeth, jaw movement such as jaw movement based on bruxism (i.e., grinding teeth), and the like.

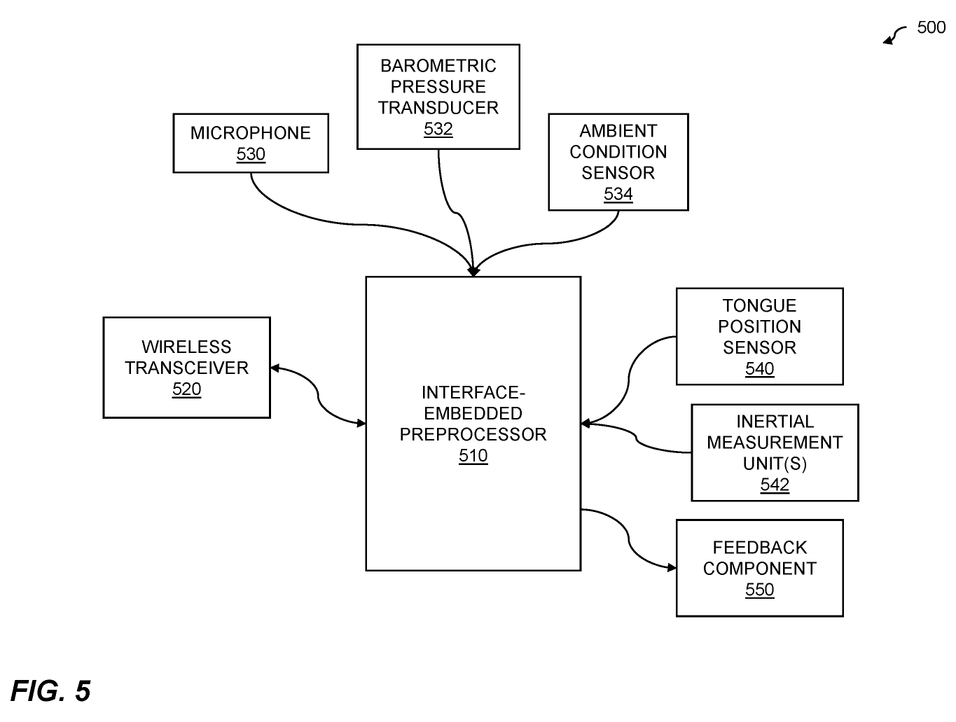

FIG. 5 is an example apparatus for context-aware gestures with an oral sensor interface. A tongue position sensor, inertial measurement units, biometric sensors, and so on, can be used to collect data from and process data for a user. The data collecting and processing enables data manipulation using context-aware gestures with an oral sensor interface. An oral sensor interface is accessed. The oral sensor interface enables wireless connectivity to an operating system running on one or more processors. The oral sensor interface enables, with the operating system, oral mouse function control. The oral sensor interface is inserted into a user’s oral cavity. An activation gesture by a user is sensed by the oral sensor interface. The activation gesture selects, within the operating system, a mouse control mode within one or more mouse control modes.

The example 500 shows an apparatus for data manipulation comprising: an oral sensor interface, wherein the oral sensor interface enables wireless connectivity to an operating system running on one or more processors, and wherein the oral sensor interface enables, with the operating system, oral mouse function control. The example 500 can include an interface-embedded preprocessor 510. The interface-embedded preprocessor can process data collected from a variety of sensors such as a tongue position sensor, an inertial measurement unit, a barometric pressure transducer, a microphone, and so on. The interface-embedded preprocessor can be coupled between a wireless transceiver and the outputs from the TPS, the IMU, and the barometric pressure transducer. The preprocessor can be used to calibrate, filter, convert, or otherwise handle data collected from the TPS, the IMUs, and the barometric pressure transducer. The preprocessor can offload any or all processing requirements from the wirelessly connected processor. The preprocessor can enable the wirelessly connected processor to be disconnected for periods of time.

The apparatus can include a wireless transceiver 520. The wireless transceiver can include a transmitter/receiver pair (TX/RX), where the wireless transceiver can enable two-way data transfer between an external device and interface-embedded devices coupled to the transceiver, including the interface-embedded preprocessor. The external device can include a computer or processor, a smart device such as a smartphone or smartwatch, a tablet computer, a laptop computer, and the like. In embodiments, the external device can be used to set up or initialize the interface, while most or all of the processing is accomplished in the preprocessor. The preprocessor can then communicate status or actions back to the external device. In such embodiments, the external device is often a smartphone or a smartphone-connected device.

The apparatus can include a variety of sensors that can be coupled to the interface-embedded processor. In embodiments, a microphone 530 can be coupled to the interface-embedded processor. The microphone can include an audio microphone, a transducer, a bone-conduction “microphone,” or another audio pickup device suitable for audio collection. The microphone can be operated “normally on,” “normally off,” etc. In embodiments, the microphone can be enabled based on an output from an interface-embedded sensor. The microphone can capture audio data, speech data, utterances, and so on. In a usage example, the microphone can enable near-silent speech recognition.

In embodiments, the oral sensor interface includes a barometric pressure transducer 532, wherein the barometric pressure transducer measures one or more pressure changes within the oral cavity of a user. The pressure changes within the oral cavity of the user can include an increase in pressure, a decrease in pressure, a duration of a pressure change, etc. The barometric pressure transducer can be coupled to the interface-embedded processor. The barometric pressure transducer can be used to determine barometric pressure within the oral cavity of the user. In embodiments, the barometric sensor can detect a multi-value state of air pressure within an oral cavity containing the interface. The multi-value state of air pressure can include nominal or ambient pressure, increased pressure, and decreased pressure. In embodiments, the multi-value state can include ambient barometric pressure, increased barometric pressure due to exhaling into a closed oral cavity, and decreased barometric pressure due to inhaling from a closed oral cavity. The barometric pressure transducer can detect a continuum of barometric pressures.

An ambient condition sensor 534 can be coupled to the interface-embedded processor. The ambient condition sensor can include one or more biometric sensors. In a usage example, the biometric sensors can include temperature, heart rate, hydration, pH, oxygen, microbe, hormone, enzyme, blood pressure, jaw clenching force, and airflow sensors.

In embodiments, the oral sensor interface includes a tongue position sensor (TPS) 540, wherein the TPS detects a tongue movement of a user. The TPS can be coupled to the interface-embedded processor (or preprocessor). The TPS can be used as a tongue-based gesture interface. The TPS can detect tongue position, tongue pressure, tongue movement, tongue direction, etc.

In embodiments, the oral sensor interface includes an inertial measurement unit (IMU) 542, wherein the IMU detects one or more head movements of a user. The IMU can be coupled to the interface-embedded processor. In a usage example, one or more IMUs can be coupled. The IMU or IMUs can be used to measure jaw position, head acceleration, head rotation, etc. The TPS, the one or more IMUs, and the barometric pressure transducer can be used to accommodate situational impairment experienced by a person. Situational impairments can include high or low volume noise, poor lighting, social constraints, legal constraints, and so on. Situational impairments can prevent a user from reading a display, interacting with a device such as a computing device, etc. A feedback component 550 can be coupled to the interface-embedded preprocessor. The feedback component can provide haptic feedback, audio feedback, and so on. In a usage example, the feedback can originate in the processor and can be transmitted to the interface using the wireless connectivity. The feedback can be provided based on a range of actions, to accomplish a variety of tasks, etc. In embodiments, the feedback can be responsive to active oral manipulation by a user of the interface, passive monitoring of a user of the interface, and the like.

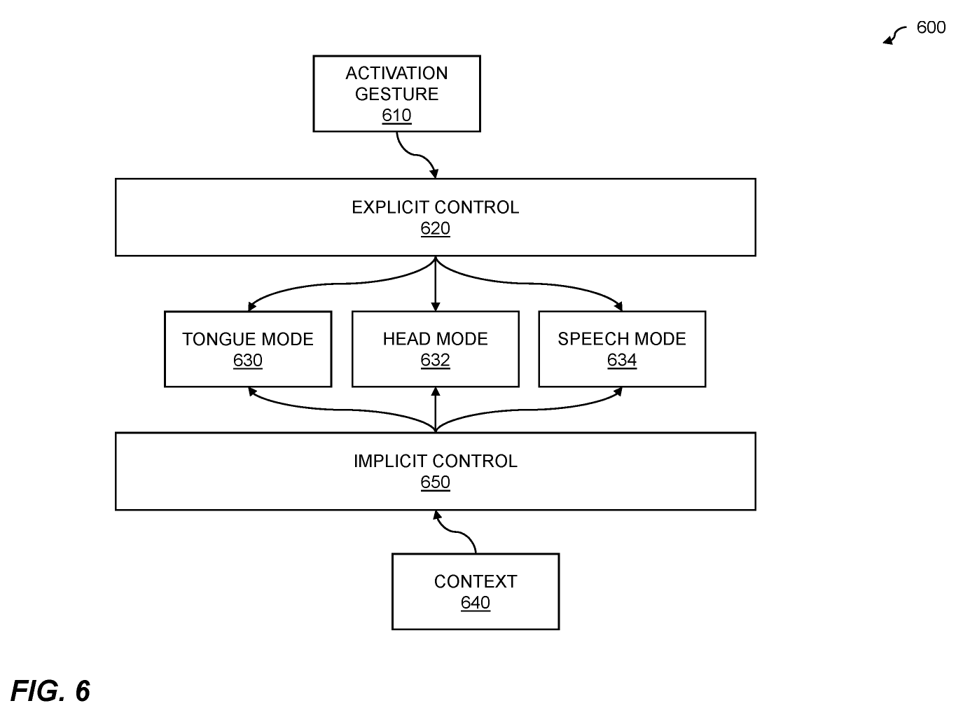

FIG. 6 is an example of controlling modes. Discussed previously and throughout, a plurality of oral gestures can be mapped to a mouse function. The mouse function can include clicking, double clicking, cursor control, selecting, swiping, and so on. The mouse function to which an oral gesture is mapped can depend on a control mode. The control modes can include a tongue mode, a head mode, a speech mode, or another mode. While functions associated with a mouse are described, the functions can further be associated with other devices such as mobility devices. In a usage example, the function can include control of a wheelchair. The function can include wheelchair velocity, wheelchair direction, wheelchair maneuvering (e.g., steering), wheelchair braking, etc. The mouse functions can be based on the control modes. That is, an activation function based on tongue pressure, a head nod, an increase in barometric pressure in the mouth, or speech can initiate different operations based on the mouse control. In a usage example, a nod of the user’s head can acknowledge a menu option, swipe, and so on. The controlling modes can enable data manipulation based on context-aware gestures with an oral sensor interface. An oral sensor interface is accessed. The oral sensor interface enables wireless connectivity to an operating system running on one or more processors. The oral sensor interface enables, with the operating system, oral mouse function control. The oral sensor interface is inserted into a user’s oral cavity. An activation gesture by a user is sensed by the oral sensor interface. The activation gesture selects, within the operating system, a mouse control mode within one or more mouse control modes.

In the example 600, a diagram of controlling modes for data manipulation is shown. An activation gesture 610 by the user can be sensed by the oral sensor interface. The activation gesture can include a tongue press, a tongue direction, a tongue movement direction, a tongue press duration, a sip, a head movement, a sound or vocalization, and so on. The controlling modes can include explicit control 620. The explicit control can be controlled by a user, by an assistant on behalf of the user, and so on. The explicit control can be based on one or more configuration parameters, where the configuration parameters can include system default parameters, user-controller parameters such as user preferences, and the like. The explicit control can be based on an application. In a usage example, a user can desire to use their oral sensor interface to manipulate a game such as a massively multiplayer online role playing game (MMORPG). The user can explicitly select configuration parameters that support playing the game. In a second usage example, the user can plan to use a suite of applications intended for work activities. The user can explicitly select configuration parameters for word processing.

The explicit control can include mapping a plurality of activation gestures to a mouse function. The mapping of gestures to a mouse function can be based on one or more mouse control modes. The example 600 includes three modes. In embodiments, the one or more mouse control modes include a tongue mode 630. The tongue mode can be based on tongue movement of the user, where the tongue movement is captured by the tongue position sensor (TPS). The TPS can detect three-dimensional tongue movements, such as up and down, left and right, movement velocity, movement direction, and so on. In embodiments, the sensing includes recognizing, by the TPS, the activation gesture, wherein the activation gesture is based on the tongue movement. The activation gesture can include a click, a double click, cursor movement, etc. In embodiments, the one or more mouse control modes include a head mode 632. The head mode can perform mapping of activation gestures to mouse functions based on head movements. Embodiments include detecting, by the inertial measurement unit (IMU), one or more head movements of the user. The user head movements can include head position, head tilt, head rotation, head nodding (e.g., up and down movement), etc. In embodiments, the one or more mouse control modes include a speech mode 634. The speech mode can include detecting by a microphone, a transducer, etc., words, phrases, and so on. The speech mode can also include detecting nonspeech utterances.

The diagram can further include a context 640. The context can include a context in which the oral sensor interface can be used to control a mouse. The context can be based on an interface application such as mouse control, accessibility device, and so on. The context can also be based on an application. In a usage example, the context can include a user who is operating a word processing application. The mouse control associated with the word processing application can include positioning a cursor, entering text, selecting text effects such as bold or italic, and so on. In a second usage example, the context can include a user playing a video game. The mouse control associated with the video game can include movement, weapon selection, weapon operation, etc. The diagram can include implicit control 650. The implicit control can include selecting from among modes such as the tongue mode, the head mode, and the speech mode, and switching among the modes based on an application such as word processing or gaming, a time of day such as working hours or nights and weekends, etc. The implicit control can be determined by an operating system, an application, etc. In a usage example, the implicit control can be based on an agent executing in the background on a processor. The agent can sense which application or app is executing and can automatically switch the mode for the user. The switching of the mode by the agent can be configured by one or more parameters such as user preferences. The implicit control can be overwritten by the explicit control.

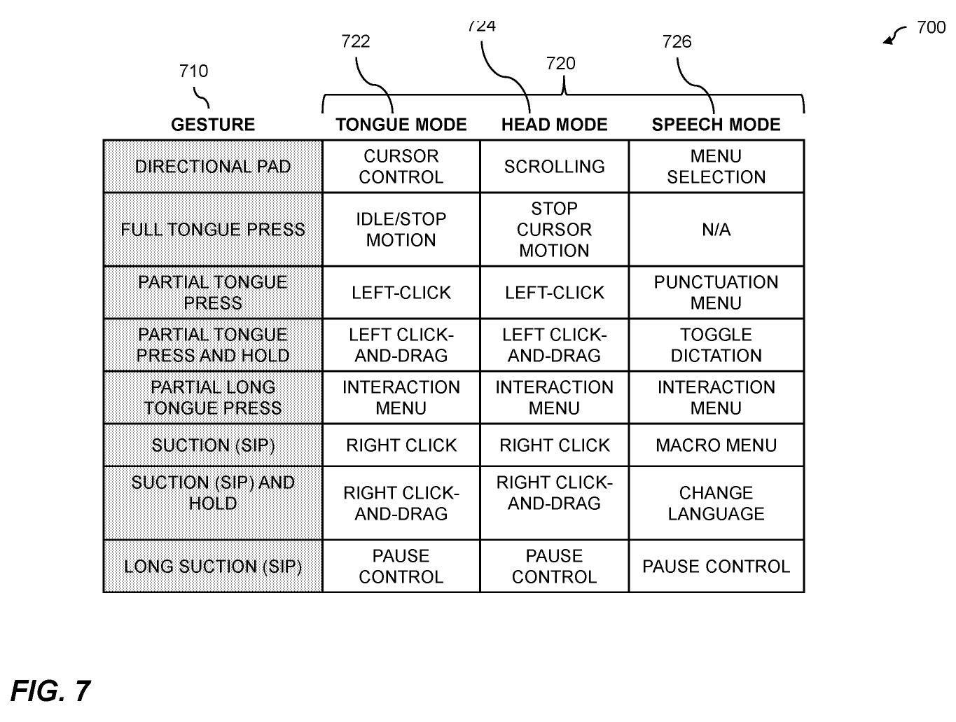

FIG. 7 in an example table of mouse control modes. Activation gestures by a user are sensed by an oral sensor interface. The activation gestures are used to operate a device such as a computer mouse. The operations that can be performed by the mouse can include clicking, double clicking, swiping, selecting, cursor control, and so on. The activation gestures can also be used to operate other devices such as mobility devices. In this latter case, the operation of the mobility devices can include direction, velocity, braking, and the like. The mouse control modes are enabled by context-aware gestures with an oral sensor interface. An oral sensor interface is accessed. The oral sensor interface enables wireless connectivity to an operating system running on one or more processors. The oral sensor interface enables, with the operating system, oral mouse function control. The oral sensor interface is inserted into a user’s oral cavity. An activation gesture by a user is sensed by the oral sensor interface. The activation gesture selects, within the operating system, a mouse control mode within one or more mouse control modes.

The activation gestures can select one or more mouse control modes within one or more mouse control modes. Thus, a single activation gesture can perform one or more operations based on the mode or context in which the activation gesture is performed. In a usage example, directional motion of the tongue on the tongue position sensor can enable cursor control in tongue mode, scrolling in head mode, and menu selection in speech mode. The mode can be selected explicitly by the user, implicitly by an app or agent executing on a processor, etc. The example table 700 includes gestures 710. The gestures can be based on position of the tongue on the TPS, a portion of the tongue in contact with the TPS, a duration of tongue contact with the TPS, and so on. The table includes modes 720. In the example table, the modes include a tongue mode 722, a head mode 724, and a speech mode 726. Other modes can be included such as a wheelchair mode (not shown in FIG. 7). In a mode such as a wheelchair mode, various gestures, such as those shown in 710, can be mapped to various control mechanisms for a wheelchair such as moving forward, moving slowly forward, moving quickly forward, moving backwards, moving slowly backwards, moving fast backwards, turning left, turning right, stop, and so on. In a usage example, in a wheelchair mode, a user can move their tongue left on the TPS which can cause a wheelchair to turn left to the same degree as the tongue movement. In another usage example, a forward movement of the tongue can cause the wheelchair to move forward at a speed that is related to how far forward the user moves their tongue on the TPS. A press on the TPS can cause the wheelchair to stop. Many combinations are possible. Additionally, many other modes can be added, including modes based on sensing barometric pressure within the user’s oral cavity; sensing user biometric data such as temperature, blood pressure, and heart rate; etc.

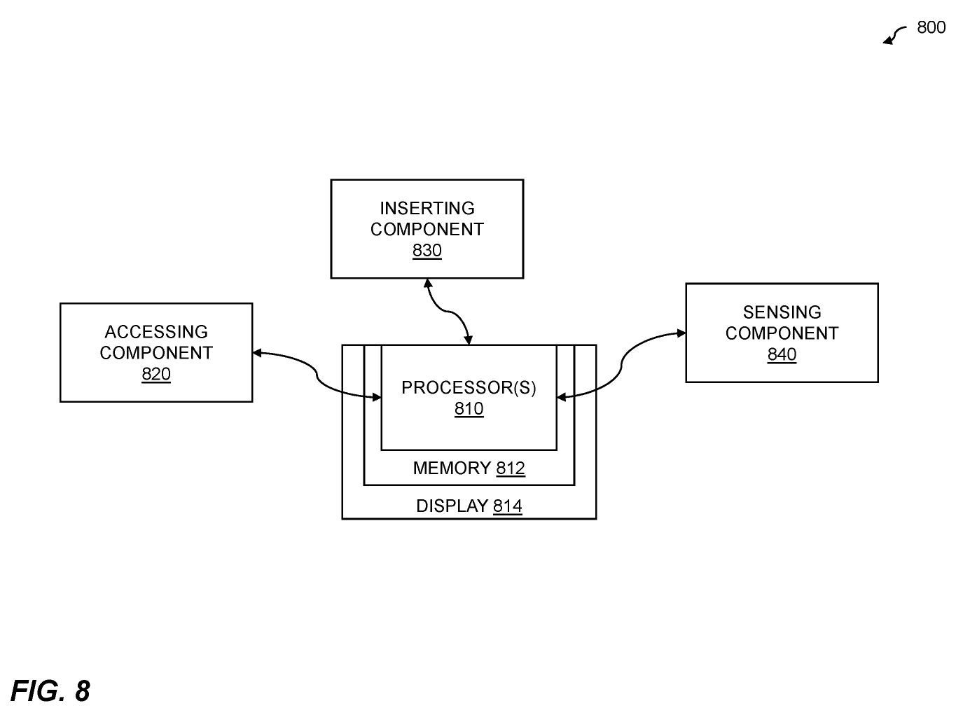

FIG. 8 is a system diagram for data manipulation using context-aware gestures with an oral sensor interface. The context-aware gestures such as activation gestures can be used for data manipulation, where the sensing of the context-aware gestures can be accomplished using a tongue position sensor (TPS), a barometric pressure transducer, an ambient condition sensor, a microphone, one or more inertial measurement units (IMUs), and so on. The sensors can be used to measure or detect a multi-value state of pressure within an oral cavity, speech including near silent speech, non-speech utterances, ambient conditions, etc. Data manipulation can further be accomplished using a prosthetic such as a retainer, where the retainer can be used to measure tongue position, tongue pressure, tongue movement, tongue direction, and the like. In a usage example, the data manipulation can include using a retainer for wireless communication, gesture input, bio-signal tracking, audio output, etc. The sensing one or more activation gestures can be used as a hands-free input and output device for data manipulation, where hands-free operation can be used by people who may not be able to use other data manipulation devices such as keyboards, mice, trackpads, smart pencils, etc. In another usage example, the hands-free operation can include hands-free operation of a mobility device such as a wheelchair.

The activation gesture sensing can be used for silent, covert, or discreet data manipulation. Wireless connectivity is provided between a processor and a wireless transceiver, where the wireless transceiver is embedded in an oral sensing interface such as a retainer, a smart-tooth implant, and the like. Data manipulation is enabled on a processor based on head movement, tongue movement, barometric pressure, speech, and so on. The system 800 can include one or more processors 810 and a memory 812 which stores instructions. The memory 812 is coupled to the one or more processors 810, wherein the one or more processors 810 can execute instructions stored in the memory 812. The memory 812 can be used for storing instructions, runtime libraries, data manipulation routines, sensor drivers, error codes or error handling routines, and so on. The memory can further be used for storing sensor calibration data. Information such as sensor data can be shown on a display 814 connected to the one or more processors 810. The display can comprise a television monitor, a projector, a computer monitor (including a laptop screen, a tablet screen, a netbook screen, and the like), a smartphone display, a mobile device, or another electronic display.

The system 800 can include a computer system for data manipulation comprising: a memory which stores instructions; one or more processors coupled to the memory, wherein the one or more processors, when executing the instructions which are stored, are configured to: access an oral sensor interface, wherein the oral sensor interface enables wireless connectivity to an operating system running on one or more processors, and wherein the oral sensor interface enables, with the operating system, oral mouse function control; insert the oral sensor interface into a user’s oral cavity; and sense, by the oral sensor interface, an activation gesture by a user, wherein the activation gesture selects, within the operating system, a mouse control mode within one or more mouse control modes.

The system 800 can include an accessing component 820. The accessing component 820 can include functions and instructions for accessing an oral sensor interface, wherein the oral sensor interface enables wireless connectivity to an operating system running on one or more processors, and wherein the oral sensor interface enables, with the operating system, oral mouse function control. The oral sensor interface can include a prosthetic device or an oral appliance such as a retainer. The oral sensor interface can include a smart tooth implant. The accessing the oral sensor interface can be accomplished using one or more wireless technologies. The wireless connectivity can be based on wireless communications standards and techniques such as 802.11 Wi-Fi, Bluetooth, near field communication (NFC), near field magnetic induction (NFMI), ZigBee, a wireless personal area network (WPAN), and so on. The wireless connectivity can include bidirectional communications capabilities. In a usage example, a wireless transceiver can be embedded in the oral sensing interface, where the oral sensing interface includes a retainer. The retainer can include sensors, contacts, communications components, and the like. The operating system running on one or more processors can include a standard operating system such as UNIX; Windows; one or the Apple operating systems such as iOS, iPadOS, and macOS; etc. The one or more processors can include handheld devices, portable devices, servers, cloud servers, and the like.

The system 800 can include an inserting component 830. The inserting component 830 can include functions and instructions for inserting the oral sensor interface into a user’s oral cavity. The inserting can be accomplished by a user inserting the oral sensor into their own oral cavity (e.g., mouth), by an assistant or medical professional inserting the oral sensor into the user’s mouth, and so on. The inserting can include inserting a retainer comprising the oral sensor interface into the user’s mouth. The oral sensor interface can include one or more sensors. In embodiments, the oral sensor interface includes a tongue position sensor (TPS). The TPS can be used to sense tongue position, tongue contact with the oral sensor interface, tongue pressure against the interface, tongue movement, tongue direction, etc. In embodiments, the oral sensor interface includes an inertial measurement unit (IMU). The IMU can be used to detect one or more head movements of the user such as head position, head tilt, head rotation, etc. In embodiments, the oral sensor interface includes a barometric pressure transducer. The barometric pressure transducer can be used to measure barometric pressure within the user’s mouth. Embodiments include detecting, by the barometric pressure transducer, a multi-value state of air pressure within the user’s oral cavity. The multi-value state of air pressure within the user’s oral cavity can include a change in barometric pressure, a pressure rate of change, and so on.

The system 800 can include a sensing component 840. The sensing component 840 can include functions and instructions for sensing, by the oral sensor interface, an activation gesture by a user, wherein the activation gesture selects, within the operating system, a mouse control mode within one or more mouse control modes. Discussed previously, an activation gesture can include tongue direction on a directional pad; a full or partial tongue press; a partial tongue press and hold; a partial long tongue press; suction (a sip); suction (sip) and hold; long suction (sip), etc. The sensing the activation gesture can perform an operation such as a mouse operation based on a mode. The activation gesture can also perform an operation associated with another device such as a mobility device. Various modes can be applied to the activation gesture to determine an action. In embodiments, the modes can include a tongue mode, a head mode, a speech mode, or another mode. Thus, a single activation gesture can perform different operations for different modes. In a usage example, a partial tongue press activation gesture can perform a left-click operation in tongue mode, a left-click operation in head mode, or can bring up a punctuation menu in speech mode.

The sensing activation gestures can be used for enabling data manipulation in a processor, based on the wireless connectivity and output from the TPS, an IMU, and a barometric pressure sensor. Other sensor data can be used to augment the TPS, the IMU, and barometric pressure transducer data such as ambient condition sensors, biometric sensors, and so on. The data manipulation can be based on acceleration, rotation, and position of the jaw; pressure exerted by the jaw, both left side and right side; and so on. The data manipulation can be further based on barometric pressure within a closed oral cavity using the barometric sensor. In embodiments, the barometric sensor can detect a multi-value state of air pressure within an oral cavity containing the interface. The tri-value state of air pressure can be based on exhaling into the closed oral cavity to increase barometric pressure, inhaling from the closed oral cavity to reduce pressure, or maintaining a neutral pressure. In embodiments, the data manipulation can be based on speech information. The speech information can include words, phrases, nonspeech utterances, and the like. The speech information can be captured using a microphone or other audio collection component coupled to a wireless transceiver. In a usage example, the microphone is enabled based on an output from an embedded sensor. For example, if tongue motion, pressure, location, etc. is detected, then the microphone can be enabled. In embodiments, the microphone enables near-silent speech recognition. The microphone can also be enabled based on an additional sensor embedded onto the interface.

A feedback component (not shown) can be associated with the system. The feedback component can provide feedback to a user of the oral sensor interface. The feedback can include haptic feedback generated by a haptic device attached to the oral sensor interface. The haptic feedback can include vibration, pressure, generating a tingling sensation, and so on. The feedback can include audio feedback generated by a sound generating device attached to the oral sensor interface. The audio feedback can include a tone or alert, speech, music, and so on. The feedback can originate in the processor and can be transmitted to the oral sensor interface using the wireless connectivity. The audio feedback can use jawbone structure for audio propagation. The feedback, whether haptic, audio, etc., can originate in the processor and can be transmitted to the oral sensor interface using wireless connectivity, where the wireless connectivity can be based on wireless connectivity techniques discussed throughout.

The system 800 can include a computer program product embodied in a non-transitory computer readable medium for data manipulation, the computer program product comprising code which causes one or more processors to perform operations of: accessing an oral sensor interface, wherein the oral sensor interface enables wireless connectivity to an operating system running on one or more processors, and wherein the oral sensor interface enables, with the operating system, oral mouse function control; inserting the oral sensor interface into a user’s oral cavity; and sensing, by the oral sensor interface, an activation gesture by a user, wherein the activation gesture selects, within the operating system, a mouse control mode within one or more mouse control modes.

Disclosed embodiments include an apparatus for data manipulation comprising: an oral sensor interface, wherein the oral sensor interface enables wireless connectivity to an operating system running on one or more processors, and wherein the oral sensor interface enables, with the operating system, oral mouse function control. The oral sensor interface includes a tongue position sensor (TPS), wherein the TPS detects a tongue movement of the user. The oral sensor interface includes an inertial measurement unit (IMU), wherein the IMU detects one or more head movements of the user. The oral sensor interface includes a barometric pressure transducer, wherein the barometric pressure transducer measures one or more pressure changes within the oral cavity of a user.

Each of the above methods may be executed on one or more processors on one or more computer systems. Embodiments may include various forms of distributed computing, client/server computing, and cloud-based computing. Further, it will be understood that the depicted steps or boxes contained in this disclosure’s flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagram and flow diagram illustrations depict methods, apparatus, systems, and computer program products. The elements and combinations of elements in the block diagrams and flow diagrams show functions, steps, or groups of steps of the methods, apparatus, systems, computer program products and/or computer-implemented methods. Any and all such functions—generally referred to herein as a “circuit,” “module,” or “system”— may be implemented by computer program instructions, by special-purpose hardware-based computer systems, by combinations of special purpose hardware and computer instructions, by combinations of general-purpose hardware and computer instructions, and so on.

A programmable apparatus which executes any of the above-mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are limited neither to conventional computer applications nor the programmable apparatus that run them. To illustrate: the embodiments of the presently claimed invention could include an optical computer, quantum computer, analog computer, or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM); an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States, then the method is considered to be performed in the United States by virtue of the causal entity.

While the invention has been disclosed in connection with preferred embodiments shown and described in detail, various modifications and improvements thereon will become apparent to those skilled in the art. Accordingly, the foregoing examples should not limit the spirit and scope of the present invention; rather it should be understood in the broadest sense allowable by law.

What is claimed is:

1. A processor-implemented method for data manipulation comprising:
accessing an oral sensor interface, wherein the oral sensor interface enables wireless connectivity to an operating system running on one or more processors, and wherein the oral sensor interface enables, with the operating system, oral mouse function control;
inserting the oral sensor interface into a user’s oral cavity; and
sensing, by the oral sensor interface, an activation gesture by a user, wherein the activation gesture selects, within the operating system, a mouse control mode within one or more mouse control modes.

2. The method of claim 1 wherein the oral sensor interface includes a tongue position sensor (TPS).

3. The method of claim 2 further comprising detecting, by the TPS, a tongue movement of the user.

4. The method of claim 3 wherein the detecting includes three-dimensional movements of the tongue of the user.

5. The method of claim 3 wherein the sensing includes recognizing, by the TPS, the activation gesture, wherein the activation gesture is based on the tongue movement.

6. The method of claim 1 wherein the oral sensor interface detects a plurality of oral gestures.

7. The method of claim 6 further comprising mapping the plurality of oral gestures to a mouse function, wherein the mapping is based on the mouse control mode.

8. The method of claim 7 wherein the one or more mouse control modes include a tongue mode.

9. The method of claim 7 wherein the one or more mouse control modes include a head mode.

10. The method of claim 7 wherein the one or more mouse control modes include a speech mode.

11. The method of claim 1 wherein the oral sensor interface includes an inertial measurement unit (IMU).

12. The method of claim 11 further comprising detecting, by the IMU, one or more head movements of the user.

13. The method of claim 12 wherein the sensing includes recognizing, by the IMU, the activation gesture, wherein the activation gesture is based on the one or more head movements of the user.

14. The method of claim 1 wherein the oral sensor interface includes a barometric pressure transducer.

15. The method of claim 14 further comprising detecting, by the barometric pressure transducer, a multi-value state of air pressure within the user’s oral cavity.

16. The method of claim 15 wherein the sensing includes recognizing, by the barometric pressure transducer, the activation gesture, wherein the activation gesture is based on a change of the multi-value state of air pressure within the user’s oral cavity.

17. The method of claim 1 wherein the enabling is based on one or more application programming interfaces (APIs).

18. The method of claim 1 wherein the sensing is based on a machine learning model.

19. A computer system for data manipulation comprising:
a memory which stores instructions;
one or more processors coupled to the memory, wherein the one or more processors, when executing the instructions which are stored, are configured to:
access an oral sensor interface, wherein the oral sensor interface enables wireless connectivity to an operating system running on one or more processors, and wherein the oral sensor interface enables, with the operating system, oral mouse function control;
insert the oral sensor interface into a user’s oral cavity; and
sense, by the oral sensor interface, an activation gesture by a user, wherein the activation gesture selects, within the operating system, a mouse control mode within one or more mouse control modes.

20. An apparatus for data manipulation comprising:
an oral sensor interface, wherein the oral sensor interface enables wireless connectivity to an operating system running on one or more processors, and wherein the oral sensor interface enables, with the operating system, oral mouse function control.

21. The apparatus of claim 20 wherein the oral sensor interface includes a tongue position sensor (TPS), wherein the TPS detects a tongue movement of a user.

22. The apparatus of claim 20 wherein the oral sensor interface includes an inertial measurement unit (IMU), wherein the IMU detects one or more head movements of a user.

23. The apparatus of claim 20 wherein the oral sensor interface includes a barometric pressure transducer, wherein the barometric pressure transducer measures one or more pressure changes within an oral cavity of a user.
